(19) United States
(12) Patent Application Publication
(10) Pub. No.: US 2012/0078529 A1
(43) Pub. Date: Mar. 29, 2012

(54) DETERMINING THE SEVERITY OF 5-FLUOROURACIL OVERDOSE

(75) Inventors: James Dennen O'Neil, Frederick, MD (US); Reid W. von Borstel, Potomac, MD (US)

(73) Assignee: WELLSTAT THERAPEUTICS CORPORATION, Gaithersburg, MD (US)

(21) Appl. No.: 13/319,563

(22) PCT Filed: May 11, 2010
(86) PCT No.: PCT/US2010/034370

§ 371 Date: Dec. 14, 2011

Related U.S. Application Data
(60) Provisional application No. 61/177,890, filed on May 13, 2009.

(51) Int. Cl. G06F 19/00; G01N 33/15

(52) U.S. Cl. 702/19

(57) ABSTRACT

A severity score for 5-fluorouracil (5FU) toxicity is calculated by taking the square root of the sum of: (a) the square of the logarithm of the dose of 5-fluorouracil administered to the patient; and (b) the square of the logarithm of the administration rate of the 5-fluorouracil to the patient.

Severity Score=[(log Dose)²+(log Rate)²]^{1/2}.

### BACKGROUND OF THE INVENTION

5-Fluorouracil (5FU) is widely used to treat solid tumors and is often administered via infusion pump at or near its maximum tolerated dose (MTD). Toxicities and even death can occur in patients over-exposed to 5FU. The use of 2′,3′,5′-tri-O-acetyluridine to treat toxicity due to 5FU is disclosed in WO 93/01202 (Pro-Neuron, Inc.). An improved method of evaluating the severity of 5FU overdoses would be desirable in order to facilitate identification of patients who could benefit from antidote treatment.

### SUMMARY OF THE INVENTION

This invention provides a method of determining a 5-fluorouracil toxicity severity score for a patient receiving 5-fluorouracil, comprising calculating the square root of the sum of: (a) the square of the logarithm of the dose of 5-fluorouracil administered to the patient; and (b) the square of the logarithm of the administration rate of the 5-fluorouracil to the patient.

This invention is based, in part, on the discovery that 5FU toxicity can be evaluated as a function of dose and infusion rate according to the method of this invention.

### DESCRIPTION OF THE FIGURES

FIG. 1: Severity score calculation. See FIG. 5 for a key of the different symbols.

FIG. 2: Vistonuridine (chemical name: 2′,3′,5′-tri-O-acetyluridine) is an oral prodrug of uridine.

FIG. 3: Fluorouracil triphosphate (FUTP) incorporation into RNA is the primary mechanism of 5FU dose-limiting toxicity.

FIG. 4: Uridine from vistonuridine is converted to uridine triphosphate (UTP), which competes with FUTP for incorporation into RNA, thereby preventing cell death and dose-limiting clinical toxicity.

FIG. 5: Observed and expected outcomes of patients receiving 5FU depending on dose and dose rate. Patients receiving 5FU at a dose and dose rate combination that is normally lethal in the absence of vistonuridine, survived when they received vistonuridine. Vertical reference line: two times the maximum dose used in a bolus regimen (1814 mg). Horizontal reference line: two times the maximum rate used in an infusion regimen (270 mg/hour). The intersection of these lines define quadrants of expected outcomes for systemic 5FU poisoning. Upper right quadrant: expected outcome in absence of vistonuridine is death. Dark gray shaded area: expected outcome in absence of vistonuridine is serious or life-threatening toxicity.

### DETAILED DESCRIPTION OF THE INVENTION

The severity score can be represented by the following equation:

Severity Score=[(log Dose)²+(log Rate)²]^{1/2}

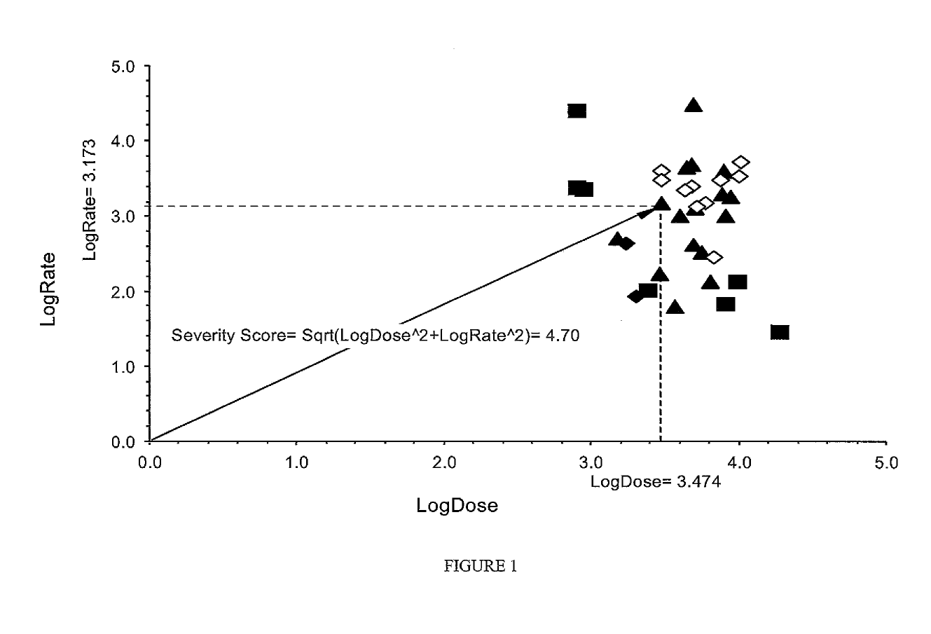

The severity score is equivalent to the distance from the origin to the observed point on the plot shown in FIG. 1. Typically the logarithm is a base-ten logarithm (log₁₀):

ti Severity Score=[(log₁₀ Dose)²+(log₁₀ Rate)²]^{1/2}

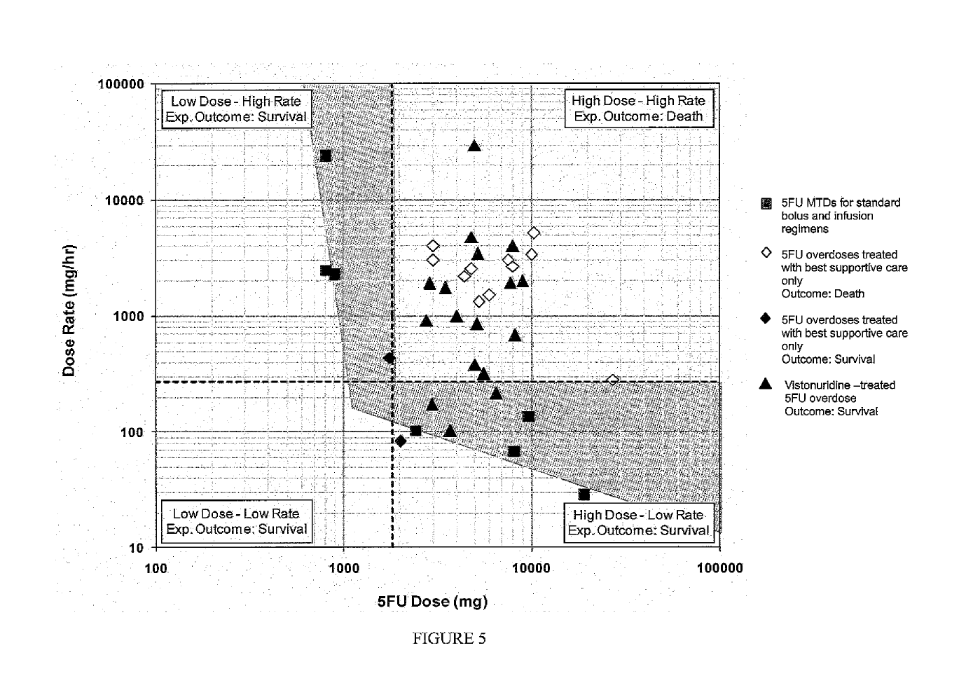

In accordance with this invention the dose and the dose rate can be expressed in any units, including all units that are conventional for expressing drug doses and dose rates. In embodiments of this invention the dose of 5-fluorouracil is expressed in units of milligrams (mg) and the rate of 5-fluorouracil administration is expressed in units of milligrams per hour (mg/hr). When the calculation is performed using base ten logarithms, the dose is expressed in units of mg, and the administration rate is expressed in units of mg/hr, then a severity score of 4.5 or higher indicates a lethal dose, and such patients should receive vistonuridine (chemical name: 2′,3′,5′-tri-O-acetyluridine; abbreviation: TAU). Patients having low severity scores do not require vistonuridine. Patients having a severity score in the intermediate range, corresponding to the dark gray area in FIG. 5 and indicating serious toxicity that is however not generally lethal, may benefit from treatment with vistonuridine. Whether they should receive vistonuridine should be evaluated on a case-by-case basis by the patient's physician. When units other than milligrams and mg/hr are utilized or a power relationship other than log₁₀ is utilized in the calculation, the severity score will have a different numerical value and the skilled artisan can readily calculate the corresponding cut-offs distinguishing among lethal, seriously toxic but nonlethal, and nontoxic, doses. The methods and calculation in accordance with this invention can be performed utilizing any conventional techniques or means for performing calculations, including a dedicated computer or a general purpose computer.

5-Fluorouracil Overdose or Overexposure:

5-Fluorouracil (5FU) is widely used for the treatment of solid tumors. Exceeding the absolute dose or infusion rate for a regimen's established maximum tolerated dose (MTD) would be expected to result in serious or life-threatening toxicity. Patients have received 5FU overdoses for various reasons, including: infusion pump malfunction or misprogramming, dose calculation errors, excess or accidental ingestion of oral 5FU sources such as capecitabine or tegafur, or administration of concomitant drugs that impair 5FU degradation. Deficits in 5FU degradation enzymes such as dihydropyrimidine dehydrogenase (DPD) can cause lethal overexposure at MTD for standard dosing regimens. In the United States about 275,000 cancer patients receive 5FU annually. The U.S. National Institutes of Health (NIH) estimates that about 3% (8250) of these patients will develop a toxic reaction and more than 1300 patients die each year from 5FU overexposure (Federal Register 73(9):38233, 2008). The symptoms of 5FU overdose typically do not appear for a few days. Therefore the ability to readily identify 5FU overdoses before symptoms of overdose are apparent is desirable.

Pharmacologic Rationale:

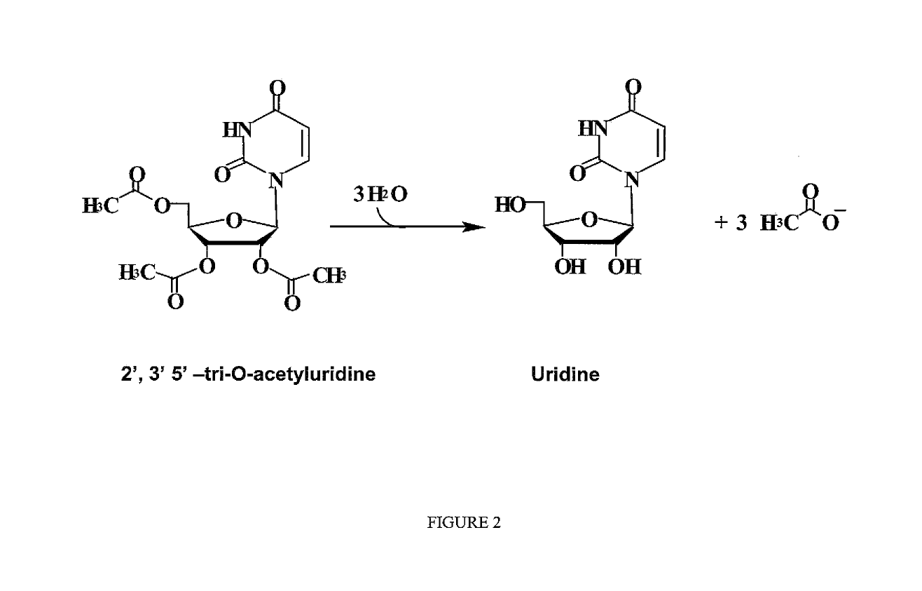

Uridine is a specific pharmacologic antidote for 5FU poisoning. It reduces 5FU toxicity when taken up prior to the onset of cell death. However uridine has poor oral bioavailability (about 7%). Vistonuridine (chemical name: 2′,3′,5′-tri-O-acetyluridine) is an oral prodrug of uridine. It is efficiently absorbed since it is more lipophilic than uridine, is not a substrate for uridine phosphorylase, and does not require transporter. Vistonuridine is rapidly converted to circulating uridine by deacetylation (FIG. 2). It is an effective antidote against 5FU poisoning when administered up to 48 hours or more after a lethal 5FU overdose in mice, and up to 96 hours or more in humans. (See Example 1 below).

Clinical Pharmacology:

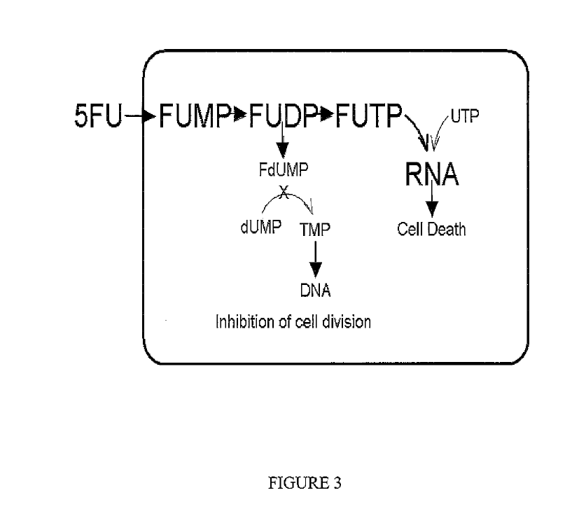

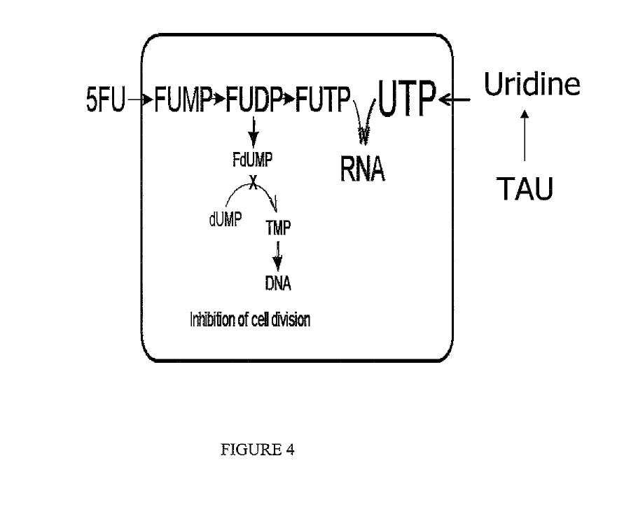

5FU is anabolized to cytotoxic intracellular intermediates. Fluorouracil triphosphate (FUTP) incorporation into RNA is the primary mechanism of dose-limiting toxicity and is proportional to systemic 5FU exposure (FIG. 3). Uridine from vistonuridine is converted to uridine triphosphate (UTP), which competes with FUTP for incorporation into RNA, thereby preventing cell death and dose-limiting clinical toxicity (FIG. 4).

Kinetics of 5-Fluorouracil:

The kinetics of 5FU are nonlinear as a function of dose. The in vivo concentration of 5FU and its toxic metabolites can increase exponentially in response to linear increases in the dose. As a result, seemingly modest overdoses can have profound toxic effects. For standard bolus regimens, 5FU is administered at lower doses and higher rates. For standard infusion regimens, 5FU is administered at higher doses and lower rates. Because 5FU toxicity, which is directly related to systemic exposure as measured by the area under the plasma concentration multiplied by time curve (AUC=area under the curve), is also a function of both dose and infusion rate, the severity score in accordance with this invention is a useful alternative to determining the AUC. And it is an easier, and therefore generally quicker, means of evaluating exposure to 5FU than determining the AUC.

### EXAMPLE

### Example 1

Methods:

Seventeen patients overdosed with 5FU have been treated with vistonuridine as an antidote. Patients received vistonuridine (10 g q6h for 20 doses) beginning 8 to 96 hours after overdose. Data from 13 patients with similar 5FU overdoses provide the time course and outcomes for patients receiving available supportive care without vistonuridine. A severity score, integrating dose and infusion rate, was calculated for all the patients, and this tool could be used by healthcare workers to determine the expected severity and outcome of a 5FU overdose.

Accidental Overdose Case Reports:

Wellstat Therapeutics Corporation was contacted by physicians of patients who had received 5FU overdoses, most due to infusion pump errors. Emergency Investigational New Drug approvals (INDs) were obtained from the U.S. Food and Drug Administration (FDA), and vistonuridine was promptly shipped or couriered to the clinics. Patients received vistonuridine beginning 8 to 96 hours after 5FU.

Control Patients—Best Supportive Care Only:

Information on doses and outcomes for 5FU overdose cases were obtained from published reports.

Results:

All 17 overdose patients treated with vistonuridine recovered fully, most with relatively modest toxicity. In marked contrast, all 11 of the literature-reported cases of 5FU overdose for which an outcome of death would have been predicted did in fact die from the overdose despite receiving available supportive care. (Table 1 and FIG. 5).

TABLE 1

| | | | |
|---|---|---|---|
| | Infusion | Calculated | Observed |
| 5FU (mg) | Duration (h) | Severity Score | Outcome |

Best Supportive Case

| | | | |
|---|---|---|---|
| 10400 | 2 | 5.47 | Death |
| 10000 | 3 | 5.33 | Death |
| 7500 | 2.5 | 5.21 | Death |
| 8000 | 3 | 5.19 | Death |
| 27200 | 96 | 5.07 | Death |
| 4800 | 1.9 | 5.01 | Death |
| 3000 | 0.75 | 5.01 | Death |
| 4400 | 2 | 4.94 | Death |
| 6000 | 4 | 4.94 | Death |
| 3000 | 1 | 4.92 | Death |
| 5250 | 4 | 4.85 | Death |
| 1750 | 4 | 4.18 | Recovered |
| 2000 | 24 | 3.82 | Recovered |

Vistonuridine Antidote

| | | | |
|---|---|---|---|
| 5000 | 0.17 | 5.80 | Recovered |
| 8000 | 2 | 5.31 | Recovered |
| 4800 | 1 | 5.21 | Recovered |
| 8960 | 4.5 | 5.15 | Recovered |
| 5180 | 1.5 | 5.13 | Recovered |
| 7720 | 4 | 5.09 | Recovered |
| 8200 | 12 | 4.83 | Recovered |
| 3472 | 2 | 4.80 | Recovered |
| 2866 | 1.5 | 4.77 | Recovered |
| 5130 | 6 | 4.73 | Recovered |
| 4000 | 4 | 4.69 | Recovered |
| 2765 | 3 | 4.54 | Recovered |
| 5000 | 13 | 4.51 | Recovered |
| 5600 | 17.5 | 4.51 | Recovered |
| 6510 | 30 | 4.47 | Recovered |
| 2940 | 17 | 4.13 | Recovered |
| 3700 | 36 | 4.10 | Recovered |

Patients received vistonuridine (10 g q6 hr for 20 doses) beginning 8 to 96 hours after the 5FU overdose

1. A method of determining a 5-fluorouracil toxicity severity score for a patient receiving 5-fluorouracil, comprising calculating the square root of the sum of: (a) the square of the logarithm of the dose of 5-fluorouracil administered to the patient; and (b) the square of the logarithm of the administration rate of the 5-fluorouracil to the patient.

2. The method of claim 1, wherein the logarithm is a base ten logarithm.

3. The method of claim 1, wherein the dose of 5-fluorouracil is expressed in units of milligrams.

4. The method of claim 1, wherein the rate of 5-fluorouracil administration is expressed in units of milligrams per hour.

5. A method of identifying a patient receiving a toxic or lethal 5-fluorouracil overdose comprising the method of claim 1.

6. The method of claim 5, wherein the logarithm is a base-ten logarithm, the dose of 5-fluorouracil is expressed in units of milligrams, the rate of 5-fluorouracil administration is expressed in units of milligrams per hour, and a severity score of 4.5 or higher indicates a lethal dose of 5-fluorouracil.
